(19) United States
(12) Patent Application Publication
(10) Pub. No.: US 2013/0345628 A1
(43) Pub. Date: Dec. 26, 2013

(54) NARROW PROFILE CATHETER WITH DEFORMATION-RESISTIVE GUIDEWIRE LUMEN

(71) Applicants: Erwin Berger, Stettfurt (CH); Youri Popowski, Geneve (CH)

(72) Inventors: Erwin Berger, Stettfurt (CH); Youri Popowski, Geneve (CH)

(73) Assignee: ACROSTAK CORP BVI,TORTOLA, Winterthur (CH)

(21) Appl. No.: 13/985,656

(22) PCT Filed: Feb. 16, 2012
(86) PCT No.: PCT/EP12/52686

§ 371 Date: Aug. 15, 2013

Related U.S. Application Data
(60) Provisional application No. 61/443,458, filed on Feb. 16, 2011.

(30) Foreign Application Priority Data
Feb. 16, 2011 (EP) 11154721.2

(51) Int. Cl. A61M 25/10

(52) U.S. Cl. CPC A61M 25/10; A61M 25/1011
USPC 604/101.05; 604/103.04

(57) ABSTRACT

The present disclosure relates to a catheter having a longitudinal guidewire lumen defined by a first tube configured for an over-the-wire or rapid-exchange mode of operation, the wall of the first tube provided with an expansion member that is a sub-region of the wall of the first tube provided with an expandable material. An inflation lumen extending from the proximal end towards the distal end of the shaft is in fluidic contact with the expansion member, the member being configured to expand or contract responsive to pressure in the inflation lumen. The wall of the first tube may be formed from a helically coiled wire or helically braided wire having a helix angle of 60 degrees or more which tubing is resistive to radial pressure applied in the inflation lumen.

### FIELD OF THE INVENTION

This invention relates to a catheter having a narrow profile suited for advancement through the vasculature over a guidewire in an over-the-wire or rapid-exchange mode of operation. More particularly, the invention relates an adaptation of the guidewire lumen to controllable release fluid, which release is modulated by an inflatable member adapted for switchable occlusion of the guidewire lumen. The guidewire lumen has a dual function, reducing the number of required lumens, and concomitantly, the profile of the catheter.

### BACKGROUND TO THE INVENTION

The use of catheters to treat structures, stenoses, or narrowings in various parts of the human body is well known in the prior art. Examples of such catheters are given in Bonzel U.S. Pat. No. 4,762,129, Yock U.S. Pat. No. 5,040,548, Kanesaka U.S. Pat. No. 5,330,499, Solar U.S. Pat. No. 5,413,557, and Tsukashima et al. U.S. Pat. No. 5,458,639.

In many cases, it is usual practice to advance a guidewire along the vessel to the region to be treated, along which a catheter can later be guided. The catheter may be used to administer a fluidic substance (medicament, dye (e.g. radio-opaque, contrast media, biochemical product, proteins or peptides, etc.) to the region of treatment, to widen a vessel by way of a balloon, and/or deploy a stent.

There are two main types of catheter in common use—rapid exchange (monorail) and over the wire (OTW). Over the wire catheters employ a long guidewire lumen from the proximal end to the distal end of the catheter; these require a guidewire of a sufficient length that the portion outside the body of an in situ guidewire is greater than the full length of the catheter to allow catheter exchange. In this mode, guidewires are often 4 m long or longer, and require a dedicated assistant to handle the wire. Despite the disadvantages, over the wire catheters are widely used for difficult lesions such as chronic total occlusions of coronary arteries where better pushability of OTW catheters is preferred, as well as in peripheral arteries where radiologists have been used mainly working with OTW catheters and where pushability may also be advantageous. Rapid exchange catheters employ a distal guidewire lumen, having a side port for exits of the guidewire towards the distal end. The fact that the guidewire is received only within a distal portion allows the catheter to be readily exchanged without the need for guidewire extenders or for an excessively long guidewire.

A catheter provided, for example, with a balloon, is required to deliver a fluidic substance to the treatment site in certain procedures. One channel of the catheter is dedicated to passage of guidewire, another channel is required for balloon inflation, and fluidic medicament is delivered through a third dedicated channel. It is a problem in the art that, some regions of vessels such as the distal peripheral vasculature below the knee, are narrow to the extent that a catheter cannot pass. Often a vessel is first widened using the guidewire, sometimes using a supporting catheter, and finally guiding the multichannel balloon catheter using the guide wire to the diseased area. The widening procedure requires time when balloon catheters have difficulties crossing the diseased area, which could be at least partially avoided by employing a narrow profile balloon catheter.

Another reason for which physicians like to have balloon catheters with a narrow profile is because it allows having less traumatic, smaller diameter introducers (short tubes allowing entering arteries at the level of the groin or wrist). Ideally these introducers should have an inner diameter of 5 or 6 F. Reduction of balloon profile (by reducing the number of its channels for instance) allows for reducing the diameter of the introducer.

In view of the foregoing, it is an object of this invention to provide an improved catheter for use with guide wires. In particular, the invention aims to provide a catheter having inner lumen walls that are resistive to deformation under inflation. In particular, the present invention aims to provide a narrow profile catheter having the ability to deliver or to aspirate fluidic substance through an existing channel without sacrificing the independent operability of each channel. This and various other objects, advantages and features of the present invention will become apparent from the following description and claims, when considered in conjunction with the appended drawings.

### SUMMARY OF SOME EMBODIMENTS OF THE INVENTION

One embodiment of the invention relates to a catheter (100) having a proximal end (20) and distal end (10), comprising:
an elongated longitudinal shaft (30), which forms the outer wall of an inflation lumen (36) extending from the proximal (20) end towards the distal (10) end of the shaft (30), and
an inner lumen (57), for the passage of fluidic substance or guidewire, disposed within the inflation lumen (36) and fluidicly isolated therefrom, wherein at least part of the wall (39) of the inner lumen (57) is made from tubing (8) reinforced with a helically coiled wire (12) or helically braided wire (14) having a helix angle of 60 degrees or more which tubing is resistive to radial pressure applied in the inflation lumen (36).

The shaft (30) may comprise at least one inflatable balloon (50) at the distal end (10), the inflation lumen (36) being in fluidic connection with a balloon lumen (52).

The wall (39) of the inner lumen (57) may comprise two regions of tubing of differential stiffness in the longitudinal direction,
an R-region containing said reinforced tubing (8), and
an F-region distal to the R-region, and containing tubing (4) that is more flexible than that in the R-region, and optionally devoid of the coiled wire (12) or braided wire (14).

The wall (39) of the inner lumen (57) may comprise a further region of tubing of differential stiffness, that is an S-region proximal to the R-region, containing tubing which is less flexible than that in the R-region, and which is optionally devoid of the coiled wire (12) or braided wire (14).

The inner lumen (57) may be a guidewire lumen (32) terminating in a distal terminal port (38) in the shaft (30), configured for an over-the-wire or rapid-exchange mode of operation, and
the wall (33) of the guidewire lumen (32) may contain an expansion member (34) that is a sub-region of the guidewire lumen (32) wall (33) provided with an expandable material,
the inflation lumen (36) may be in fluidic contact with the expansion member (34), said member (34) configured to expand or contract responsive to pressure in the inflation lumen (36), thereby switchably occluding the guidewire lumen (32).

The catheter may further comprise a transverse lumen (TL, 40), proximal (20) to the expansion member (34), fluidicly connecting the guidewire lumen (32) to a TL-side port (41) on the side wall of the shaft (30). The expansion member (34) may be located distal to the TL-side port (41). The guidewire lumen (32), configured for an over-the-wire mode of operation, may extend to the proximal terminal end (20) of the flexible shaft (30).

The guidewire lumen (32) of the catheter may alternatively be configured for a rapid exchange mode of operation, is branched, wherein
a side branch (32′) is provided for the passage of a guidewire through a GL (guidewire) side port (46) in side wall of the shaft (30)
a longitudinal branch (32″) extends to the proximal (20) terminal end of the shaft (30), configured for the passage of fluid, to the exclusion of the guidewire.

The catheter may comprise an additional expansion member (44), said additional expansion member (44) located on the side branch (32′).

The catheter (100—FIG. 24) may further comprise:
a guidewire lumen (32) extending from the open proximal end of the shaft (30) to a distal terminal port (38) in the shaft (30), disposed within the inflation lumen (36) and fluidicly isolated therefrom,
a transverse lumen (TL, 40), fluidicly connecting the guidewire lumen (32) to a TL-side port (41) on the side wall of shaft (30),
   wherein
the wall (33) of the guidewire lumen (32) is provided with an expansion member (34) that is a sub-region of the guidewire lumen (32) wall provided with an expandable material;
the inflation lumen (36) is in fluidic contact with the expansion member (34), said member (34) configured to expand or contract responsive to pressure in the inflation lumen (36), thereby switchably occluding the guidewire lumen,
the transverse lumen (TL, 40) is proximal (20) to the expansion member (34),
the inner lumen is an additional lumen (35—FIG. 24) being
   fluidicly isolated from the inflation lumen (36) and the guidewire lumen (32),
   concentrically arranged around part of the guidewire lumen (32) proximal (20) to the TL side port (41) and to the expansion member (34).
   closed and fluidicly sealed against the outer wall (33) of the guidewire lumen (32) at its distal end
   connected via an additional transverse lumen (ATL, 43) to at least one ATL-side port (45) on the side wall of the shaft (30).

The ATL side port (43) of the catheter, when it is a balloon catheter, may be located proximal to the most proximal inflatable balloon (50). The number of inflatable balloons (50, 50′) may be two or more, and any two balloons (50, 50′) flank an ATL side port (43).

The catheter (100—FIG. 23) may further comprise:
a guidewire lumen (32) terminating in a distal terminal port (38) in the shaft (30), configured for a rapid exchange mode of operation, disposed within the inflation lumen (36) and fluidicly isolated therefrom,
   wherein
the inner lumen (57) is an additional lumen (35) being
   fluidicly isolated from the inflation lumen (36) and the guidewire lumen (32),
   concentrically arranged around part of the guidewire lumen (32),
   closed and fluidicly sealed against the outer wall (33) of the guidewire lumen (32) at its distal end, and
   connected via an additional transverse lumen (ATL, 43) to at least one ATL-side port (45) on the side wall of the shaft (30).

The proximal end of the guidewire lumen (32), may terminate in a GL (guidewire) side port (55) in the side wall of the shaft (30), towards the distal end of the shaft (30).

Where the catheter is a balloon catheter, the TL side port (41) may be located proximal to the most proximal inflatable balloon (50). Where the number of inflatable balloons (50, 50′) is two or more, any two balloons (50, 50′) may flank a TL side port (41).

Another embodiment of the invention is a catheter (100) having a proximal end (20) and a distal end (10), comprising an elongated flexible shaft (30) containing:
a longitudinal guidewire lumen (32) terminating in a distal terminal port (38) in the shaft (30), said guidewire lumen (32) configured for an over-the-wire or rapid-exchange mode of operation, the wall of the guidewire lumen provided with an expansion member (34) that is a sub-region of the guidewire lumen (32) wall provided with an expandable material; and
an inflation lumen (36) extending from the proximal (20) end towards the distal (10) end of the shaft (30) and in fluidic contact with the expansion member (34), said member (34) configured to expand or contract responsive to pressure in the inflation lumen (36).

The catheter may further comprise a transverse lumen (TL, 40), proximal (20) to the expansion member (34), fluidicly connecting the longitudinal guidewire lumen (32) to a TL-side port (41) on the side wall of the shaft (30).

The expansion member (34) of the catheter may be located distal to the TL-side port (41).

The shaft (30) may comprise an inflatable balloon (50) at the distal end (10), the inflation lumen (36) being in fluidic connection with the balloon lumen (52).

The catheter may further comprise two or more inflatable balloons (50) at the distal end (10), any two of which flank a TL side port (41).

The TL side port (41) may be located proximal to the most proximal inflatable balloon (50).

The shaft (30) may comprise an IL (inflation lumen) side port (42) in fluid connection with the inflation lumen (36), which IL-side port (42) is configured to allow the passage of fluid when the pressure in the inflation lumen (36) exceeds that required to contract the expansion member (34) to occlusion of the guidewire lumen (32).

The guidewire lumen (32), may be configured for an over-the-wire mode of operation, in which case it extends to the proximal terminal end of the shaft (30).

The guidewire lumen (32) may be configured for rapid exchange mode of operation, in which case it is branched,
a side branch (32′) provided for the passage of a guidewire through a GL (guidewire) side port (46) in side wall of the shaft (30)
a longitudinal branch (32″) extending to the proximal (20) end of the shaft (30), configured for the passage of fluid, to the exclusion of the guidewire.

There may be an additional expansion member (44), said additional expansion member (44) located on the side branch (32′).

The elongated flexible shaft (30) preferably forms a wall of an inflation lumen (36) and the guidewire lumen (32) is disposed within the inflation lumen (36).

Another embodiment of the invention is a catheter (100—FIGS. 1-6) having a proximal end (20) and a distal end (10), comprising an elongated shaft (30) containing:
a longitudinal first tube (31) provided with a guidewire lumen (32) terminating in a distal terminal port (38) in the shaft (30), said guidewire lumen (32) configured for an over-the-wire or rapid-exchange mode of operation, the wall of the first tube (31) provided with an expansion member (34) that is a sub-region of the first tube (31) wall provided with an expandable material;
an inflation lumen (36) extending from the proximal (20) end towards the distal (10) end of the shaft (30) and in fluidic contact with the expansion member (34), said member (34) configured to expand or contract responsive to pressure in the inflation lumen (36); and
a transverse lumen, TL, (40) defined by an transverse tube, TT (31′), proximal (20) to the expansion member (34), fluidicly connecting the guidewire lumen (32) to a TL-side port (41) on the side wall of the shaft (30).

The shaft (30) may comprise at least one inflatable balloon (50) at the distal end (10), the inflation lumen (36) being in fluidic connection with a balloon lumen (52). The inflation lumen (36) may be defined by a second tube (29) that is the shaft (30). The TL side port (41) may be located proximal to the most proximal inflatable balloon (50), or the number of inflatable balloons (50, 50′) may be two or more and any two balloons (50, 50′) flank a TL side port (41). The expansion member (34) may be located distal to the TL-side port (41). The guidewire lumen (32), configured for an over-the-wire mode of operation, may extend to the proximal terminal end (20) of the flexible shaft (30).

At least part of the wall of the first tube (31) may be made from tubing (8) reinforced with a helically coiled wire (12) or helically braided wire (14) having a helix angle of 60 degrees or more which tubing is resistive to radial pressure applied in the inflation lumen (36). The wall of the first tube (31) may comprise two regions of tubing of differential stiffness in the longitudinal direction, an R-region containing said reinforced tubing (8), and an F-region distal to the R-region, and containing tubing (4) that is more flexible than that in the R-region, and optionally devoid of the coiled wire (12) or braided wire (14). The wall of the first tube (31) may comprise a further region of tubing of differential stiffness, that is an S-region proximal to the R-region, containing tubing that is less flexible than that in the R-region, and is optionally devoid of the coiled wire (12) or braided wire (14).

The guidewire lumen (32), configured for a rapid exchange mode of operation, may be branched, a side branch (32′) may be provided for the passage of a guidewire through a GL (guidewire) side port (46) in side wall of the shaft (30), and a longitudinal branch (32″) may extend to the proximal (20) terminal end of the shaft (30), configured for the passage of fluid, to the exclusion of the guidewire. The catheter may comprising an additional expansion member (44), said additional expansion member (44) being located on the side branch (32′).

The catheter (100—FIG. 24) may further comprise an additional inner lumen (35—FIG. 24) defined by a tube third tube (47), wherein the additional inner lumen (35) is fluidicly isolated from the inflation lumen (36) and the guidewire lumen (32), concentrically arranged around part of the guidewire lumen (32) proximal (20) to the TL side port (41) and to the expansion member (34), closed and fluidicly sealed, at its distal end, against the outer wall (33) of the guidewire lumen (32); and connected via an additional transverse lumen ATL, (43) to at least one ATL-side port (45) on the side wall of the shaft (30). The wall (37) of the third tube (47) may comprise two regions of tubing of differential stiffness in the longitudinal direction, an R-region containing said reinforced tubing (8), and an F-region distal to the R-region, and containing tubing (4) that is more flexible than that in the R-region, and optionally devoid of the coiled wire (12) or braided wire (14). The wall of the third tube (47) comprises a further region of tubing of differential stiffness, that is an S-region proximal to the R-region, containing tubing that is less flexible than that in the R-region, and is optionally devoid of the coiled wire (12) or braided wire (14).

### LEGENDS TO THE FIGURES

FIG. 1 is a schematic illustration of a catheter with a switchably occluding guidewire lumen, configured for over-the-wire operation.

FIG. 2 is a schematic illustration of a catheter with a switchably occluding guidewire lumen, configured for rapid exchange (monorail) operation.

FIG. 3 is a schematic illustration of a catheter with a switchably occluding guidewire lumen, configured for rapid exchange (monorail) operation, and provided with a transverse lumen (TL) side port.

FIG. 4 is a schematic illustration of an over-the-wire catheter with a switchably occluding guidewire lumen, provided with an inflatable balloon, and a side port proximal to the balloon.

FIG. 5 is a schematic illustration of an over-the-wire catheter with a switchably occluding guidewire lumen, provided with a plurality of balloons, and a side port between the balloons.

FIG. 6 is a schematic illustration of an over-the-wire catheter with a switchably occluding guidewire lumen, provided with an inflatable balloon, and a side port distal to the balloon.

FIGS. 7 and 8 depict a transverse (B-B′) cross-section of a catheter wherein the lumens are coaxial (FIG. 7) or adjacent (FIG. 8).

FIGS. 9 to 12 depict an operation of the catheter with a switchably occluding guidewire lumen. In FIG. 9, inflation medium is introduced through the inflation lumen; in FIG. 10 the inflatable balloon expands; FIG. 11 shows occlusion effected by the expandable member and in detail in FIG. 11a; FIG. 12 depicts the introduction of medicament through the guidewire lumen for administration through transverse lumen side ports.

FIG. 13 depicts a pair of fluid delivery couplings one for inflation of the balloons and the other for the delivery of fluidic substance, attached to the proximal end of a catheter of the invention.

FIG. 14 depicts a catheter of the invention attached to a pair of fluid delivery couplings, which in turn are each connected to a fluid pump.

FIG. 15 depicts a single device incorporating a pair of fluid delivery couplings in rigid connection.

FIG. 15a depicts the plan view of the device of FIG. 15.

FIG. 16 is a schematic illustration of a catheter comprising a reinforced inner tube according to an embodiment of the invention.

FIG. 17 is a schematic illustration of an inner tube provided with a coiled reinforcing wire.

FIG. 18 depicts the helix angle, beta, of a coiled reinforcement.

FIG. 19 is a schematic illustration of an inner tube provided with a braided reinforcing wire.

FIG. 20 depicts the helix angle, beta, of a braided reinforcement.

FIG. 21 is a schematic illustration of a catheter with a single balloon, provided with a switchably occluding guidewire lumen, configured for over-the-wire operation in which part of the guidewire lumen is formed from reinforced tubing.

FIG. 22 is a schematic illustration of a catheter with a two balloons, provided with a switchably occluding guidewire lumen, configured for over-the-wire operation in which part of the guidewire lumen is formed from reinforced tubing.

FIG. 23 is a schematic illustration of a catheter having three concentrically arranged lumens, configured for rapid exchange (monorail) operation, in which the outer wall of part of the middle lumen is formed from reinforced tubing.

FIG. 24 is a schematic illustration of a catheter having three concentrically arranged lumens, configured over-the-wire operation, in which the outer wall of part of the middle lumen is formed from reinforced tubing.

### DETAILED DESCRIPTION OF THE INVENTION

Unless defined otherwise, all technical and scientific terms used herein have the same meaning as is commonly understood by one of skill in the art. All publications referenced herein are incorporated by reference thereto. All United States patents and patent applications referenced herein are incorporated by reference herein in their entirety including the drawings.

The articles “a” and “an” are used herein to refer to one or to more than one, i.e. to at least one of the grammatical object of the article. The recitation of numerical ranges by endpoints includes all integer numbers and, where appropriate, fractions subsumed within that range (e.g. 1 to 5 can include 1, 2, 3, 4 when referring to, for example, a number of articles, and can also include 1.5, 2, 2.75 and 3.80, when referring to, for example, measurements). The recitation of end points also includes the end point values themselves (e.g. from 1.0 to 5.0 includes both 1.0 and 5.0).

The terms “comprising”, “comprises” and “comprised of” as used herein are synonymous with “including”, “includes” or “containing”, “contains”, and are inclusive or open-ended and do not exclude additional, non-recited members, elements or method steps. It will be appreciated that the terms “comprising”, “comprises” and “comprised of” as used herein comprise the terms “consisting of”, “consists” and “consists of”.

The terms “distal”, “distal end”, “proximal” and “distal end” are used through the specification, and are terms generally understood in the field to mean towards (proximal) or away (distal) from the surgeon side of the apparatus. Thus, “proximal (end)” means towards the surgeon side and, therefore, away from the patient side. Conversely, “distal (end)” means towards the patient side and, therefore, away from the surgeon side.

In the present description of the invention, reference is made to the accompanying drawings that form a part hereof, and in which are shown by way of illustration only of specific embodiments in which the invention may be practiced. Parenthesized or emboldened reference numerals affixed to respective elements merely exemplify the elements by way of example, with which it is not intended to limit the respective elements. It is to be understood that other embodiments may be utilised and structural or logical changes may be made without departing from the scope of the present invention. The following detailed description, therefore, is not to be taken in a limiting sense, and the scope of the present invention is defined by the appended claims.

The present invention concerns a catheter having a proximal end and distal end, comprising an elongated longitudinal shaft comprising a guidewire lumen and inflation lumen therewithin. The guidewire lumen is contained within a first hollow tube, while the inflation lumen is contained within a second hollow tube. The first tube may be an inner tube residing within the inflation lumen of the second tube. The guidewire lumen terminates in a distal terminal open port in the shaft, and is configured for an over-the-wire or rapid-exchange mode of operation. According to one embodiment of the invention, the first tube disposed with the guidewire lumen is provided with an expansion member that is a sub-region of the wall of the first tube formed from an expandable material. The expansion member is configured to occlude partially or fully the guidewire lumen in the expanded state, whether the guidewire is present in the guidewire lumen or not. An inflation lumen extends longitudinally from the proximal end towards the distal end of the shaft, and is in fluidic contact with the expansion member, said member configured to expand or contract responsive to pressure in the inflation lumen. The inflation lumen extends from the proximal end to a position distal to expansion member, and is sealed at its distal end.

Both the inflation lumen and guidewire lumen configured according to the invention provide a double function, reducing the requirement for additional channels, and thus narrowing the catheter profile. The inflation lumen can be employed not only to inflate an expandable balloon where present on the catheter, but also to control movement (expansion or contraction) of the expansion member. The guidewire lumen principally serves to carry the guidewire, but can also be employed for the passage of a fluidic substance introduced through the proximal end, which fluid may be a medicament, dye (e.g. radio-opaque, contrast media), biochemical product etc. The expansion member, when in an expanded state, occludes the guidewire lumen, most preferably to prevent passage of fluid through the guidewire port, re-directing fluid through a side port to at the site, for example, of the stenosed region.

By utilising the inflation lumen and guidewire lumen so, a two lumen guidewire catheter can be used to deliver fluid to a distal side port, which functionality would normally require a dedicated third lumen. As a consequence, the number of lumens is reduced, accompanied by an inevitable reduction in catheter profile. Catheters that are contemplated include, but are not limited to, cannulas, sphincterotomes, infusion catheters when mixing substances at a distal situation is necessary, such as during vertebroplasty procedures (mixing a polymer and a polymerization substance), cytology devices, and devices for stone retrieval and stent placement. The system may also be used to deliver chemotherapy such as doxorubicin microspheres to selected arteries, without the drug passing into neighbouring arteries. The infusion may be done distally or proximally to the balloon.

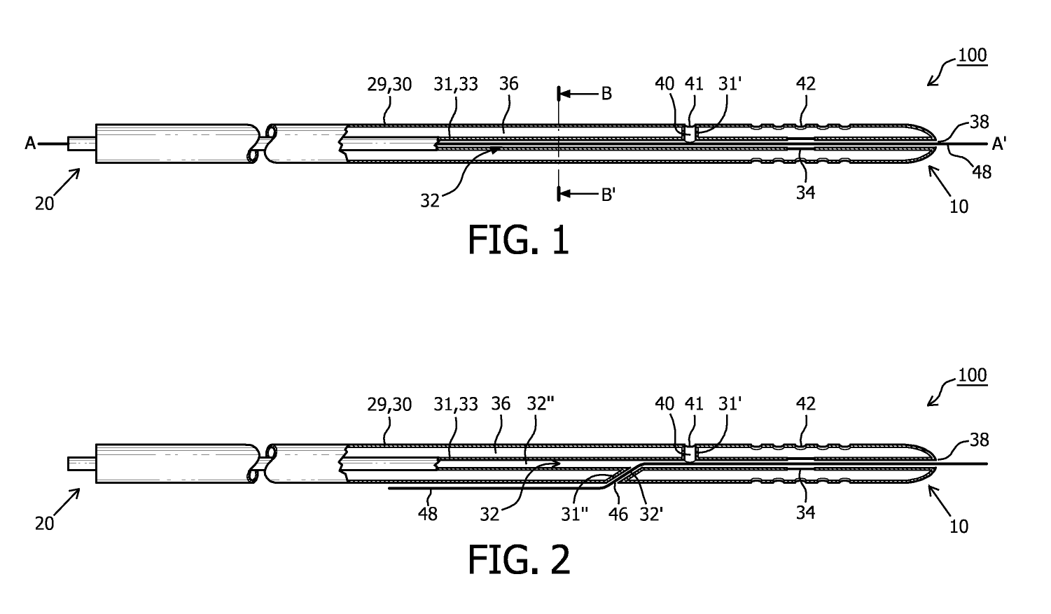

With reference to FIG. 1, one embodiment of the present invention concerns a catheter 100 comprising an elongated flexible shaft 30 having a proximal end 20, a distal end 10, an inflation lumen 36, a longitudinal guidewire lumen 32 disposed within the shaft 30. The longitudinal guidewire lumen 32 is defined by a first (inner) tube 31. The inflation lumen 36 is defined by a second (outer) tube 29, which may be the catheter shaft 30, The first (inner tube) 31 is disposed within the inflation lumen 36. For the purpose of the description herein, the catheter has longitudinal axis (A-A′) and a transverse plane (B-B′) perpendicular thereto. The distal 10 end of the elongated flexible shaft 30 terminates in a distal terminal port 38, with which the guidewire lumen 32 is in fluid connection. When catheter is provided for an over-the-wire mode of operation, as shown in FIG. 1, the guidewire lumen 32 further extends to a proximal terminal end 20 of the catheter 100. The first tube 31 is provided with an expansion member 34 that is a sub-region of the wall 33 of first tube 31 wall 33 disposed with an expandable material configured to occlude, partially or fully, the guidewire lumen 32 in the expanded state. An inflation lumen is further provided within the shaft 30, extending from the proximal end 20 to a position distal 10 to the expansion member. The inflation lumen 36 is sealed at its distal end, and is in fluidic contact with the expansion member 34. The expansion member 34 is configured to expand or contract responsive to hydraulic pressure applied to the inflation lumen 36. A fluid-delivery coupling may be attached to the proximal end of the catheter, which coupling has a guidewire port, and two lateral ports, one for introduction of inflation medium into the inflation lumen 36 and the other for infusion and/or aspiration of fluidic substance into the guidewire lumen 32. Other details of the catheter are given elsewhere in the description.

FIG. 2 presents an alternative embodiment of the invention, wherein the catheter 100 is provided for a rapid-exchange mode of operation. As with the embodiment in FIG. 1 catheter 100 comprises an elongated flexible shaft 30 having a proximal end 20, a distal end 10, and a longitudinal guidewire lumen 32 defined by a first (inner) tube 31 disposed within the lumen 36 of a second tube 29 that is the shaft 30. The distal end of the elongated flexible shaft 30 terminates in a distal terminal port 38, with which the guidewire lumen 32 is in fluid connection. The guidewire lumen 32 is branched at the distal end 10; a side branch 32′ is provided for the passage of a guidewire through a GL (guidewire lumen) side port 46 in the shaft 30 and a longitudinal branch 32″ extends to the proximal 20 end of the shaft 30, configured for the passage of fluid, to the exclusion of the guidewire. The wall 33 of the first tube 31 is provided with an expansion member 34 that is a sub-region of the first tube 31 wall formed from an expandable material configured to occlude, partially or fully, the guidewire lumen 32 in the expanded state. An inflation lumen is further disposed within the shaft 30, extending from the proximal end 20 to a position distal 10 to the expansion member. The inflation lumen 36 is sealed at its distal end, and is in fluidic contact with the expansion member 34. The expansion member 34 is configured to expand or contract responsive to hydraulic pressure applied to the inflation lumen 36. The wall of the shaft 30 may form the outer wall of the inflation lumen 36 as shown in FIG. 2. A fluid delivery coupling may be attached to the proximal end 20 of the catheter, which coupling has a guidewire port, and two lateral ports, one for introduction of inflation medium into the inflation lumen 36 and the other for infusion of fluidic substance into the guidewire lumen 32. Other details of the catheter are given elsewhere in the description.

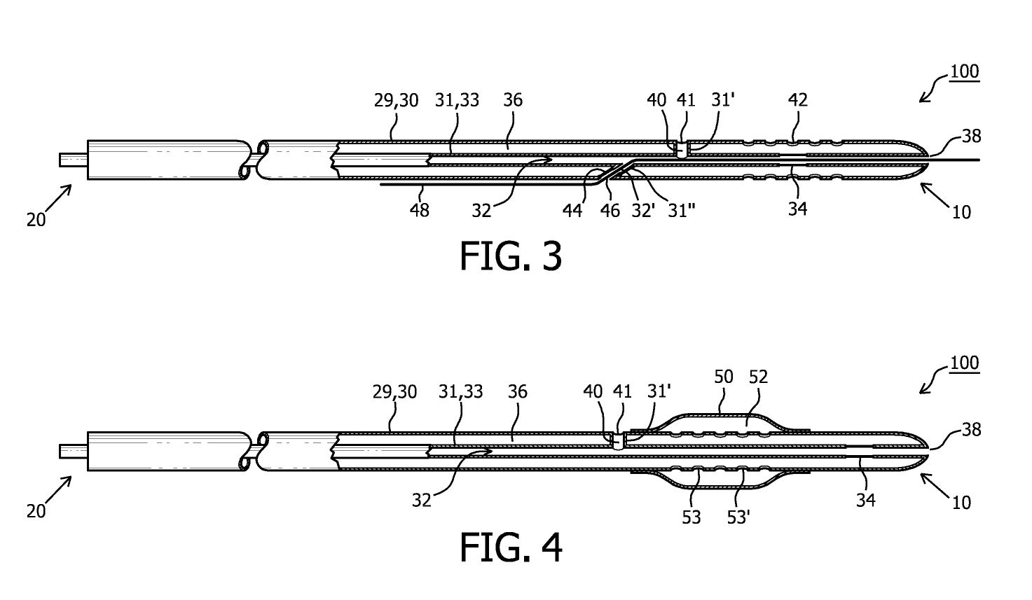

FIG. 3 presents an alternative embodiment of the invention, wherein the catheter 100 is provided for a rapid-exchange mode of operation as indicated in FIG. 2, except an additional expansion member 44 is provided on the wall of the first tube 31, more specifically at the side branch 32′. Other details of the catheter are given elsewhere in the description.

FIG. 4 illustrates an embodiment of the over-the-wire catheter provided with an inflatable balloon 50 at the distal end 10, the inflation lumen 36 being in fluidic connection with the balloon lumen 52. It will be understood that the inflatable balloon 50 is configured to expand at low hydraulic pressure that also expands and closes the expansion member 34 over the guidewire lumen 34. While FIG. 4 depicts a balloon catheter configured for an over-the-wire mode of operation, it is within the scope of the invention that the balloon catheter is alternatively configured for rapid-exchange i.e. is provided with a side branch as shown in FIG. 2 or FIG. 3. Other details of the catheter are given elsewhere in the description.

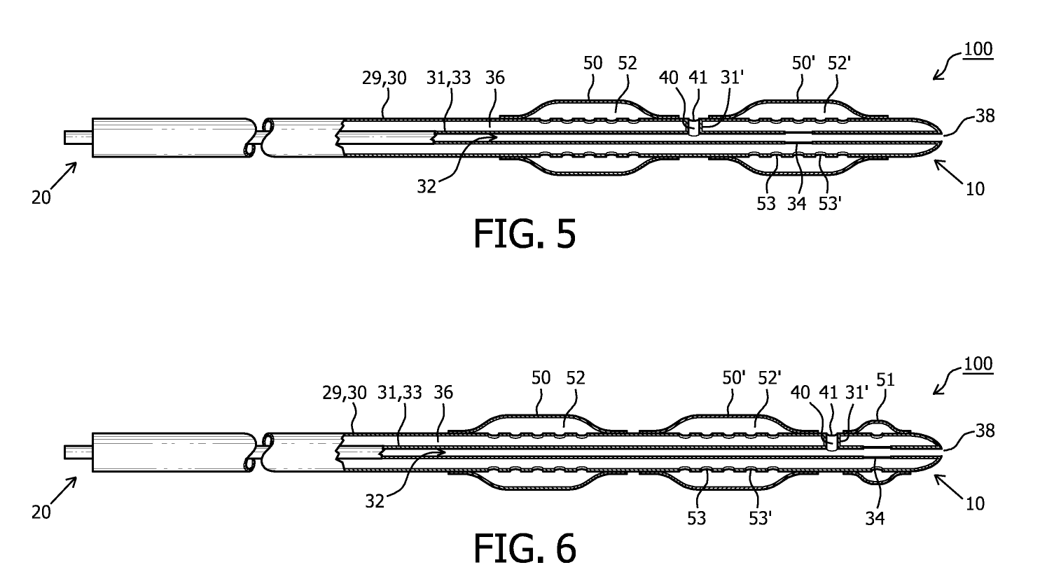

FIG. 5 illustrates an embodiment of the over-the wire catheter provided with a plurality of inflatable balloons 50, 50′ tandemly arranged at the distal end 10, the inflation lumen 36 being in fluidic connection with each balloon lumen 52. In this embodiment, each pair of adjacent balloons flanks a TL (transverse lumen) side port 41 as described elsewhere herein. While FIG. 5 depicts a multi-balloon catheter configured for an over-the-wire mode of operation, it is within the scope of the invention that the balloon catheter is alternatively configured for rapid-exchange i.e. is provided with a side branch as shown in FIG. 2 or FIG. 3.

FIG. 6 illustrates an embodiment of the over-the wire catheter provided with a plurality of inflatable balloons 50, 50′, 51 tandemly arranged at the distal end 10, the inflation lumen 36 being in fluidic connection with each balloon lumen 52. The balloons are comprised in two major balloons 50, 50′ adjacently arranged, and a minor balloon 51 arranged distal to the major balloons 50, 50′ and configured to inflate radially to a shorter distance compared with the major balloons 50.

In this embodiment, the TL (transverse lumen) side port 41, described elsewhere herein, is provided distal to the major balloons 50, 50′ and proximal to the minor balloon 51. This compared with FIG. 4 where the TL side port is proximal to the balloon 50, and FIG. 5 where the TL side port is between the balloons 50, 50′. While FIG. 6 depicts a multi-balloon catheter configured for an over-the-wire mode of operation, it is within the scope of the invention that the balloon catheter is alternatively configured for rapid-exchange i.e. is provided with a side branch as shown in FIG. 2 or FIG. 3.

It is noted that in all configurations, the expansion member 34 is located distal to the most distal TL side port 41.

When the fluid inside the inflation lumen 36 exercises a pressure on the expansion member 34, the guide wire lumen is sealed to prevent a loss of pressure. This is done automatically in the case of a rapid exchange system, where 2 expansion members (FIGS. 3, 34 and 44) are closed simultaneously by the pressure exercised inside inflation lumen 36. In the case of an OTW catheter, the compartment is closed at the distal end by the expansion member 34, and at the proximal end 20, by a fluid delivery coupling, described elsewhere herein, fixed on the proximal luer connector, or combined with the luer connector.

The catheter 100 comprises an elongated shaft 30 (also referred to as a shaft herein) having a proximal end 20 and a distal end 10. The shaft 30 may form the wall of an inflation lumen 36 i.e. the shaft 30 may be the second (outer) tube 29. Within the shaft 30 lumen is disposed a longitudinal guidewire lumen 32 defined by the first (inner) tube 31 configured for an over-the-wire or rapid-exchange mode of operation. The proximal 20 terminal ends of both the inflation 36 and guidewire 32 lumens are open (not sealed) for the passage of guidewire, and of fluidic substances such as inflation medium and medicament respectively. The guidewire lumen may be fluidicly connected to a hemostatic valve, allowing closing the guidewire compartment from both sides. The distal end 10 of elongated shaft 30 terminates in a distal terminal port 38 to which the guidewire lumen is in fluidic connection. The elongated shaft 30 is tubular, typically cylindrical, having a generally uniform outer shape in the proximal region. One or more hubs such as a Y-type connector, optionally with Luer fittings may be fitted to the proximal terminal end of the shaft to facilitate passage of the guidewire, and coupling of the catheter to equipment for providing inflation fluid and fluidic substances to the guidewire lumen. Such a hub may be a fluid delivery coupling as described elsewhere herein, which includes hemostatic valve as described, for instance, in U.S. Pat. No. 5,195,980 and which is incorporated herein by reference.

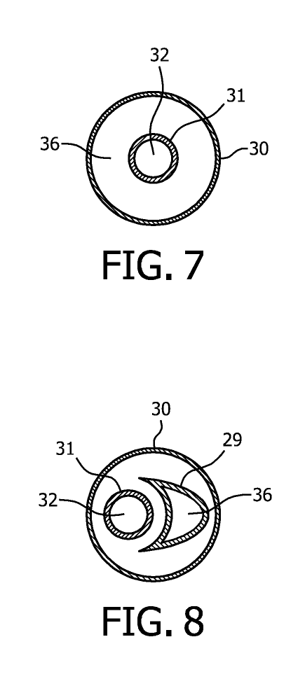

The guidewire 32 and inflation 36 lumens may be arranged within the shaft 30 in a substantially co-axial alignment (FIG. 7) with the inflation lumen 36 surrounding the guidewire lumen 32, or in a substantially side-by-side configuration (FIG. 8). Alternatively, expressed, the first tube 31 and second tube 29 may be arranged in a substantially co-axial alignment (FIG. 7) with the second tube 29 that is the shaft 30 surrounding the first tube 31, or in a substantially side-by-side configuration with the second tube 29 next to the first tube 31 and both enclosed by the shaft 30 (FIG. 8).

The elongated shaft 30 may incorporate a distal tip, through which the guidewire lumen 32 extends. The distal tip may be softened and atraumatic.

As would be understood by those of skill in the art, the shaft 30 may preferably be sized for slidable passage through, for example, the working channel of an endoscope or through a body lumen, in particular vasculature (through an introducer). As a general guidance, for vascular applications, the maximum outer diameter of the shaft 30 towards the distal (in situ) end may be equal to or no greater than 3 F (1 mm), 4 F (1.35 mm), 5 F (1.67 mm), 6 F (2 mm), 7 F (2.3 mm), 8 (2.7 mm), 9 (3 mm), 10 (3.3 mm), 11 (3.7 mm), 12 (4 mm), a value in the range between any two of the aforementioned values, preferably between 4 F and 8 F. For other applications, such as treatment of the oesophagus, or upper airway, it will be appreciated that the maximum outer diameter may be according greater without detriment.

The shaft 30 may be formed using an extrusion process or non-extrusion process. A shaft 30 may be formed from a biocompatible material which provides the requisite flexibility, pushability and strength. Suitable biocompatible materials include, but are not limited to a polymer such as polypropylene, polyethylene, polyurethanes, polyamide, polyimide poly(ethylene terephthalate) (PET) or polyesters and copolymers thereof, metal (stainless steel, nitinol) of a combination of metal and polymer. In a preferred embodiment it is formed from a polymeric material that is polyamide, polyimide, stainless steel or nitinol or a combination or blend of these. The shaft may be formed from a polymeric material (e.g. polyimide) strengthened with braided or coiled metal (stainless steel or nitinol) disposed within the polyimide wall. For a shaft formed by extrusion, it is preferably formed from polyamide. For a shaft formed by non-extrusion, it is preferably formed from polyimide. The exterior may be coated to reduce friction during insertion or withdrawal. Example of a suitable friction-reducing coating includes Teflon.

The first tube 31 may be provided with an expansion member 34 that is a sub-region of the wall 32 of the first tube 31 disposed with an expandable material. The expansion member is configured to occlude partially or fully the guidewire lumen 32 in the expanded state. The expansion member 34 is configured to move (expand or contract) responsive to hydraulic pressure applied to the inflation lumen 36 which lumen 36 is in fluidic contact with the expansion member 34.

The expansion member may be any suitable shape, for instance a circular patch, a C-shaped or O-shaped ring. The ring is preferably co-axial with the longitudinal axis (A-A′) of the guidewire lumen 32. The expansion member 34, when a ring, is provided to expand radially inwards and seal around the guidewire lumen 32 whether the guide wire 48 is present inside the guide wire lumen or not. The expansion member 34 is preferably located towards the distal end 10 of the shaft 30, most preferably in close proximity to the distal terminal port 38. It is preferably located distal to the GL side port 46 or TL side port 41 described below. The expansion member may be located in the region of a balloon or distally to the balloon for instance. Where the guidewire lumen 32 is configured for use in the over-the-wire mode, expansion member 34 is provided to occlude the passage of fluidic substance through the distal terminal port 38; in the occluded state fluidic substance can exit the catheter through a side port (e.g. the GL side port 46 or TL side port 41 described below). Where the guidewire lumen 32 is configured for use in rapid-exchange mode, a further expansion member 34 may be provided in the wall of guidewire lumen side branch 32′ provided to occlude the passage of fluid material therethrough.

The expansion member 34 is formed from any suitable expandable material capable of expansion and contraction (i.e. elastic expansion) responsive to the application of hydraulic pressure. It is a compliant member, returning to its original shape after pressure has been removed. It is elastic, i.e. elastically expandable. Examples of suitable materials include latex rubber, polyurethane, polyamide, polyolefin and any known in the art. It will be obvious that the elastic expansion of the expansion member 34 is greater than the elastic expansion of the shaft 30, or the wall 33 of the first tube 31.

Hydraulic pressure is applied to the expansion member 34 via the inflation lumen 36. Where the catheter is disposed with at least one balloon, the hydraulic pressure required to expand the expansion member 34 may be of the same order of magnitude as that required to inflate the balloon 50. The balloon expansion and the sealing effect of the expansion member 34, therefore, arise simultaneously. The balloon expansion gives the surgeon the option of whether to utilise the guidewire lumen 32 as a fluid-delivery lumen or not, after balloon 50 deployment.

The expansion member is fixed to the proximal and distal parts of the guidewire lumen; it can be welded, glued, heatshrinked with the proximal and distal parts of the guidewire lumen, or fixed by any other technique known by someone skilled in the art.

A longitudinal guidewire lumen 32 is disposed within the shaft 30 of the catheter 100 and is fluidicly isolated from the inflation lumen 36. The longitudinal guidewire lumen 32 is defined by a first tube 31. At its distal end 10, the guidewire lumen 32 terminates in a distal terminal port 38, with which the guidewire lumen 32 is in fluid connection. When catheter is provided for an over-the-wire mode of operation, as shown in FIG. 1, the guidewire lumen 32 further extends to the proximal 20 end of the shaft 30. The guidewire lumen 32 is open at its proximal end; it may be attached to one or more hubs such as a Y-type connector, optionally with Luer fittings to facilitate passage of the guidewire, and coupling to equipment for providing fluidic substances to the guidewire lumen. Such a hub may be a fluid delivery coupling as described elsewhere herein, which includes hemostatic valve as described, for instance, in U.S. Pat. No. 5,195,980 and which is incorporated herein by reference. In one embodiment of the invention, when the wall of the shaft 30 forms the outer wall of the inflation lumen 36, the longitudinal guidewire lumen 32 is disposed within the inflation lumen 36.

When the catheter is provided for a rapid-exchange operation, as shown in FIGS. 2 and 3, the guidewire lumen 32 is branched at the distal end 10; a side branch 32′ is provided for the passage of a guidewire through a GL (guidewire lumen) side port 46 in the shaft 30 configured for the passage of fluid. A rapid-exchange mode of operation as indicated in FIG. 3, shows an additional expansion member 44 provided on the wall 32 first tube 31, more specifically at the side branch 32′. The wall 32 of the first tube 31 is provided with an expansion member 34 described above that is a sub-region of the wall 32 of the first tube 31 provided with an expandable material configured to occlude, partially or fully, the guidewire lumen 32 in the expanded state.

The guidewire lumen 32 may be connected via a transverse lumen, TL, 40, to at least one (e.g. 2, 3, 4, 5, 6, 7, 8, 10 or more) TL-side port 41 on the side wall of the shaft 30 (FIGS. 1, 3 to 5, 8 to 11). The TL 40 is defined by a tube, a transverse tube, TT, 31′. The TL-side port 41 is located proximally to the expansion member 34. It is preferably positioned towards the distal 10 end of the catheter 100. Transverse lumen, TL, is essentially radial to the longitudinal axis of the catheter. The TL lumen is fluidicly isolated from the inflation lumen 36. The TL-side port 41 provides an outlet for fluidic substances introduced through the guidewire lumen 32 after the expansion member 34 has expanded and occluded the distal end of the lumen 32. Where one or more balloons 50 are disposed on the shaft 30, a TL-side port is preferably located between any two balloons 50, 50′, preferably between each and every pair of adjacent balloons. In such arrangement, fluid medicament may be delivered, for example, to an area of stenosed region sealed between two balloons, preventing rapid systemic circulation of the medicament.

It is noted that the guidewire lumen side branch 32′ may be disposed with an expansion member 44 when the guidewire lumen 32 is configured for rapid-exchange operation (FIG.

3). In other words the first tube 31, configured for rapid-exchange operation, is provided with a side branch 31″ that may be disposed with an expansion member 44. In such a configuration, the TL-side port 41 provides an outlet for a fluidic substance introduced via the guidewire lumen 32 at the proximal end. Alternatively, the side branch 31″ may be devoid of any expansion member 44; in such case the GL-side port 46 (FIG. 3) described elsewhere herein achieves the same function as the TL-side port 41. The diameter of TL-side ports 41 may be adapted according to the viscosity of the fluidic substance to be injected (e.g. the diameter may be increased with increased viscosity of the fluidic substance to be injected). The guidewire lumen side branch 32′ is fluidicly isolated from the inflation lumen 36.

The wall 33 of first tube 31, side branch 31″ and/or TT 31′ may be each be formed from any suitable material, that may be the same material or different. The material should be essentially non-expandable under hydrostatic pressure. Where the first tube 31 is in co-axial alignment with the inflation lumen, it should maintain its shape to allow passage of the guidewire and fluidic substance. Suitable materials include, but are not limited to a polymer such as polypropylene, polyethylene, polyurethanes, polyimide poly(ethylene terephthalate) (PET) or polyesters and copolymers thereof, metal (stainless steel, nitinol) of a combination of metal and polymer. In a preferred embodiment it is formed from a polymeric material that is polyimide, stainless steel or nitinol or a combination or blend of these. The lumen may be formed from a polymeric material (e.g. polyimide) strengthened with braided or coiled material (e.g. PEEK, stainless steel or nitinol) disposed within the polyimide wall. The interior may be coated to reduce friction of the guidewire. Example of a suitable friction-reducing coating includes Teflon.

According to one aspect of the invention at least part, preferably all, of the wall 33 of the first tube 31 is made from tubing 8 which is reinforced. The reinforced tubing 8 reduces deformation of the wall 33 of the first tube 31 when hydrostatic pressure is applied to the inflation lumen 36 thereby maximizing the transverse cross-sectional area of the guidewire lumen 32 for the passage of fluid or guidewire. The tubing 8 is reinforced using a coiled wire 12 (FIG. 17) or a braided wire 14 (FIG. 19) disposed in the longitudinal direction of the tubing. The coil has a helical path; it may be formed from a single helix or more than one helix. The braiding typically has a cross-cross pattern, formed from two helices of wire running in opposite directions. The inventors have surprisingly found that the helical reinforcing wire is most effective when it adopts a helix angle, beta, of 60 deg or more. Preferably, at least one strand of the coil or braiding has a helix angle of 60 deg or more. Advantageously, the thickness of the wall can be reduced compared with non-reinforced tubing. Since the hydrodynamic resistance created by a catheter lumen decreases with square of the cross-sectional area, a small increase in area has a large impact on performance. Thus, the reinforced tubing allows a significant improvement in inflation and deflation properties, while maintaining a low profile. Moreover, the improvements are maintained even when radial hydrostatic pressure is applied to the tubing, for instance, during inflation of a balloon.

The wall of the tubing 8 is made from any suitable material polymeric material such as polyamide or polyimide, preferably polyimide. The reinforcing coiled or braided wire may be made from any material having suitable tensile strength such as stainless steel, phynox, nitinol, silver, etc. The wire of the coil or braiding is provided within the wall lumen, or on the outside or inside of the wall. The reinforced tubing may be prepared by depositing the polymeric material over the coiled or braided wire; deposition allows a more accurate control over the thickness of the reinforced tube wall. The helix angle, beta, is the angle between the helix and the longitudinal (central) axis of the tubing as shown, for example, in FIGS. 18 and 20. The helix angle, beta, may be 60, 65, 60, 75, 80, 85, 90, 95 deg or more, or a value in the range between any two of the aforementioned values, preferably more than 60 deg, more preferably between 60 and 90 deg. Examples of commercially available reinforced tubing include for instance polyimide coiled tubes produced by Microlumen.

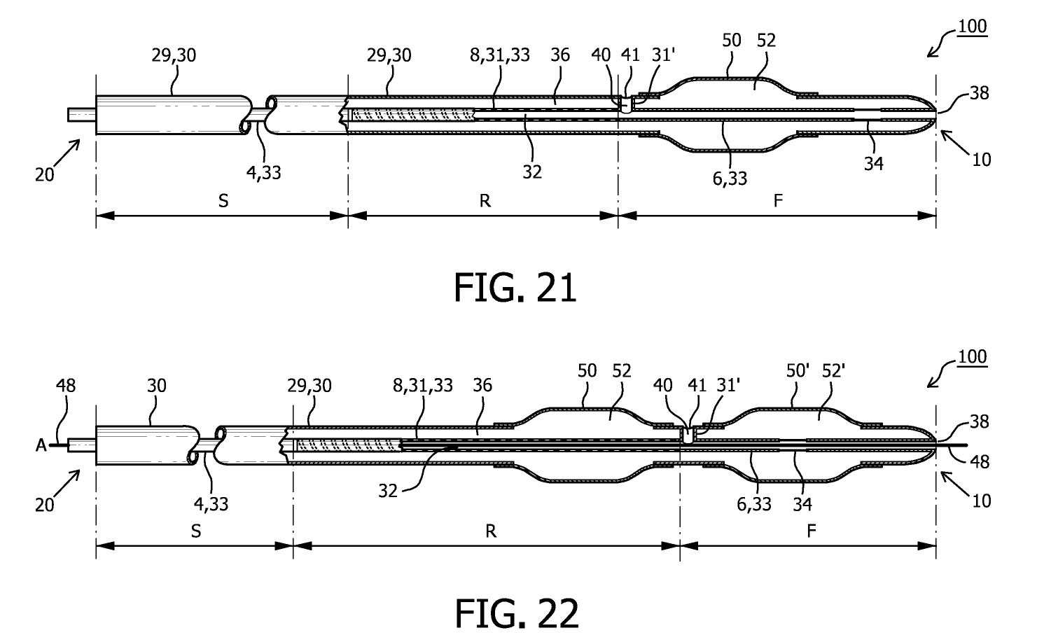

All or part of the first tube 31, 31′ wall 33 may be formed from the same reinforced tubing 8. Where it is formed in part, preferably the longitudinal part is proximal to the TL side port 40. According to one aspect of the invention, the first tube 31, is formed from three different tubing materials longitudinally arranged, giving rise to an “S” (stiffer) region at the proximal end, a “R” (reinforced) region distal to the S region and proximal to the TL side port 40, and an “F” (flexible) region distal to the R region, as illustrated, for example, in FIGS. 21 and 22. FIG. 21 illustrates an embodiment of the over-the wire catheter provided with an inflatable balloon 50 at the distal end 10, the inflation lumen 36 being in fluidic connection with the balloon lumen 52, similar to the embodiment depicted in FIG. 4. FIG. 22 illustrates an embodiment of the over-the wire catheter provided with a plurality of inflatable balloons 50, 50′ tandemly arranged at the distal end 10, the inflation lumen 36 being in fluidic connection with each balloon lumen 52 similar to the embodiment depicted in FIG. 5.

In the S region, the first tube 31 wall 33 need not be reinforced and may be made from tubing 4 that does not have a coiled or braided wire; the wall of the S region is sufficiently thick to withstand pressure applied to the inflation lumen and is generally thicker compared with the tubing in the R or F regions. The tubing in the S region may be made from any suitable material, including, but are not limited to a polymer such as polypropylene, polyethylene, polyurethanes, polyamide, polyimide poly(ethylene terephthalate) (PET) or polyesters and copolymers thereof, metal (stainless steel, nitinol) of a combination of metal and polymer. The overall catheter profile in the S region is necessarily larger to resist hydrostatic forces, thus it is used in the proximal part of the catheter that will not enter a narrowed vessel or tortuous vascular route. Typically the thickness of the wall in the S region is 50 to 150 μm, preferably 60-100 μm. When made from metal, this region may have a wall thickness similar to the thickness of the reinforced region R, however its flexibility is inferior when compared to the reinforced region R.

In the R region, the first tube 31 wall 33 is made from tubing 8 that is reinforced with a coil or braiding as described above, and is more flexible than the tubing in the S region. Typically the thickness of the wall in the R region is 30 to 100 μm, preferably 50 μm. The R-region may occupy a fraction of the total length of the catheter that is 0.2, 0.3, 0.4 or 0.5, 0.8 of the total catheter length, or a value between any two of the aforementioned values. This reinforced region R combines high resistance to pressure and great flexibility, allowing for cross-over catheterization, allowing entering through one femoral artery and going to the contro-lateral femoral artery, passing through aortic bifurcation while still allowing for guide wire passage and drug infusion.

In the F region, the first tube 31 wall 33 is not reinforced and is made from tubing 6 that does not have a coiled or braided wire. Typically the thickness of the wall in the F region is 30 to 120 μm, preferably 50 μm.

Its location distal of the TL side port 40 implies that deformation or collapse of the wall in the F region does not affect the passage of fluid that travels in the guidewire lumen proximal to the TL side port 40. As a consequence, the guidewire lumen 32, 32′ wall may be more flexible in the F region than in the R region. The tubing in the F region may be made from any suitable material, including, but are not limited to a polymer such as polypropylene, polyethylene, polyurethanes, polyamide, polyimide poly(ethylene terephthalate) (PET) or polyesters and copolymers thereof, metal (stainless steel, nitinol) of a combination of metal and polymer.

The regions of tubing adjacent the R region may be joined thereto using an adhesive.

The guidewire lumen 32 and where present, the side branch lumen 32′, (and hence first tube 31 and side branch 31′) are typically cylindrical. It is dimensioned to receive a guidewire. It will be understood that the diameter of the guidewire lumen 32, 32′ of the first tube 31 and side branch 31′ where present will depend on the diameter of the guidewire, but as a general guidance, it will be suitable for accommodating a guidewire having a diameter of 0.01″ (0.0254 cm), 0.012″ (0.03048 cm), 0.014″ (0.03556 cm), 0.018″ (0.04572 cm), or 0.02″ (0.0508 cm).

An inflation lumen 36 extends longitudinally from the proximal 20 end towards the distal 10 end, and is in fluidic contact with the expansion member 34, said member configured to expand or contract responsive to pressure (hydraulic or gaseous) in the inflation lumen. The inflation lumen is defined by the second tube 29, which may be the catheter shaft 30. The inflation lumen extends from the proximal end to a position distal 10 of the distal-most expansion member 34, and is sealed at its distal 34 end. The inflation lumen 36 is open at its proximal end; it may be attached to one or more hubs such as a Y-type connector mentioned easier, optionally with Luer fittings to facilitate coupling to equipment for providing inflation medium and fluidic substances to the inflation lumen 36. Such a hub may be a fluid delivery coupling as described elsewhere herein, which includes hemostatic valve as described, for instance, in U.S. Pat. No. 5,195,980 and which is incorporated herein by reference. When one or more inflatable balloons 50 are present on the shaft 30, the inflation lumen 36 is in fluidic connection with the balloon lumens, typically via one or more openings in the lumen wall. The guidewire lumen 32, 32′ may be cylindrical, or another shape (e.g. oval-, moon-shaped).

When inflation lumen is not in co-axial alignment with the guidewire lumen, it may have its own wall, which, may be formed from any suitable material such as metal, polyimide, polyamide, PEEK, metal, and other materials known by any skilled in the art.

The inflation lumen 36 may be connected to at least one (e.g. 2, 3, 4, 5, 6, 7, 8, 10 or more) IL (inflation lumen)-side port 42 on the shaft 30. This embodiment is typically applicable in the absence of a balloon as exemplified in FIGS. 1 to 3. The IL-side port 42 is generally located towards the distal 10 end of the shaft 30. The IL-side port 42 provides an outlet for fluidic substances introduced through the inflation lumen 36 after the expansion member 34 has expanded and occluded the distal end of the lumen 32. Thus, IL-side port 42 may be configured to allow passage of fluidic substance after the hydrostatic pressure in the inflation lumen 36 exceeds that required to expand the expansion member 34 to occlusion. It will be appreciated that the IL-side port 42 can be utilised for introducing a second fluidic substance, for instance, medicament, dye (e.g. radio-opaque, contrast media), biochemical product etc. in addition to the fluidic substance introduced through the guidewire lumen 32. Thus allows two substances to be mixed in situ for greater efficacy, and/or the treatment by sequential medicaments. The diameters of side ports 41 and 42 may be adapted to the viscosity of the products to be injected (diameter may increase along with increased viscosity of the substance to be injected).

According to one aspect of the invention, within the lumen 36 of the shaft 30 is disposed an additional lumen 35, fluidicly isolated from the inflation lumen 36 and the guidewire lumen 32 as depicted, for instance, in FIG. 24. The additional lumen 35 is defined by a tube, a third (inner) tube, 47. The additional lumen 35 or third tube 47 is essentially concentrically arranged around part of the guidewire lumen 32 proximal 20 to the TL side port 41 and to the expansion member 34. The outer wall 37 of the third tube 47 is in fluidic contact with the inflation lumen 36. At its distal end 10, the additional lumen 35 is closed and fluidicly sealed against the outer wall 33 of the guidewire lumen 32. The additional lumen 35 extends to the proximal end of the shaft 30, and is open at its proximal end 20; it may be attached to one or more hubs such as a Y-type connector, optionally with Luer fittings to facilitate passage of the guidewire, and coupling to equipment for providing fluidic substances to the additional lumen 35.

The additional lumen 35 may be connected via an additional transverse lumen, ATL, 43, to at least one (e.g. 2, 3, 4, 5, 6, 7, 8, 10 or more) ATL-side port 45 on the side wall of the shaft 30 (FIG. 24). The ATL 43 is defined by an additional transverse tube, ATT, 48.

The ATL-side port 45 is preferably positioned towards the distal 10 end of the catheter 100. The ATT 48 and associated ATL 43 are essentially radial to the longitudinal axis of the catheter. The ATL 43 is fluidicly isolated from the inflation lumen 36. The TL-side port 45 provides an outlet for fluidic substances introduced through the additional lumen 35. Where one or more balloons 50, 50′ is disposed on the shaft 30, an ATL-side port 45 is preferably located between any two balloons 50, 50′, preferably between each and every pair of adjacent balloons. In such arrangement, fluid medicament may be delivered, for example, to an area of stenosed region sealed between two balloons, preventing systemic circulation of the medicament. When combined with the TL-side port 41, two separate medicaments may be delivered simultaneous and mixed in situ. This catheter configuration may also be used to rinse a vessel segment, for instance to aspirate the thrombolysis products by dilating balloons 50 and 50′, blocking the blood flow and isolating a vessel segment: a thrombolytic agent may be infused through lateral port 41 and the thrombolysis products may be reaspirated through lateral port 45.

In addition to what has been previously described for first tube 31 and side branch 31′ in a monorail or over-the-wire conformation, according to one aspect of the invention at least part, preferably all, of the wall 37 of the third tube 47 is made from reinforced tubing 8. The reinforced tubing 8 reduces deformation of the wall 37 of the third tube 47 when hydrostatic pressure is applied to the inflation lumen 36 thereby maximizing its transverse cross-sectional area for the passage of fluid or guidewire. The tubing 8 is reinforced using a coiled wire 12 (FIG. 17) or a braided wire 14 (FIG. 19) disposed in the longitudinal direction of the tubing. The coil has a helical path; it may be formed from a single helix or more than one helix. The braiding typically has a cross-cross pattern, formed from two helices of wire running in opposite directions. The inventors have surprisingly found that the reinforcing wire is most effective when it adopts a helix angle, beta, of 60 deg or more. Preferably, at least one strand of the coil or braiding has a helix angle of 60 deg or more. Advantageously, the thickness of the wall can be reduced compared with non-reinforced tubing. Since the hydrodynamic resistance created by a catheter lumen decreases with square of the cross-sectional area, a small increase in area due to thinner catheter walls, will have a large impact on performance. Thus, the reinforced tubing will allow a significant improvement in inflation and deflation properties, while maintaining a low catheter profile.

The wall of the tubing 8 is made from any suitable material polymeric material such as polyamide or polyimide, preferably polyimide. The reinforcing coiled or braided wire may be made from any material having suitable tensile strength such as stainless steel, phynox, nitinol, silver, etc. The wire may be provided as a single strand or as a plurality of strands twisted together for additional strength. The wire may have a circular, oblong (rectangular or square), oval or rounded oblong profile. The wire of the coil or braiding is provided within the wall lumen, or on the outside or inside of the wall. The reinforced tubing may be prepared by depositing the polymeric material over the coiled or braided wire; deposition allows a more accurate control over the thickness of the reinforced tube wall. The helix angle, beta, is the angle between the helix an the central axial of the tubing as shown, for example, in FIGS. 18 and 20. The helix angle, beta, may be 60, 65, 60, 75, 80, 85, 90, 95 deg or more, or a value in the range between any two of the aforementioned values, preferably more than 60 deg, more preferably between 60 and 90 deg. Examples of commercially available reinforced tubing include for instance polyimide coiled tubes produced by Microlumen.

All or part of the third tube 47 wall 37 may be formed from the reinforced tubing 8. Where it is formed in part, preferably the part is proximal to the TL side port 40. According to one aspect of the invention, the wall 37 of the third tube 47 is formed from three different types of tubing in longitudinal arrangement giving rise to an “S” (stiffer) region at the proximal end, a “R” (reinforced) region distal to the S region and proximal to the TL side port 40, and an “F” (flexible) region distal to the R region, as illustrated, for example, in FIG. 24. FIG. 24 illustrates an embodiment of the over-the-wire catheter provided with a plurality of inflatable balloons 50, 50′ tandemly arranged at the distal end 10, the inflation lumen 36 being in fluidic connection with each balloon lumen 52 similar to the embodiment depicted in FIG. 5.

In the S region, the wall 37 of the third tube 47 may not be reinforced and may be made from tubing 4 that does not have a coiled or braided wire; the wall of the S region is sufficiently thick to withstand pressure applied to the inflation lumen and is generally thicker compared with the tubing in the R or F regions. The tubing in the S region may be made from any suitable material, including, but are not limited to a polymer such as polypropylene, polyethylene, polyurethanes, polyamide, polyimide poly(ethylene terephthalate) (PET) or polyesters and copolymers thereof, metal (stainless steel, nitinol) of a combination of metal and polymer. The overall catheter profile in the S region is necessarily larger to resist hydrostatic forces, thus it is used in proximal part 20 of the catheter that will not enter a narrowed vessel or tortuous vascular route. Typically the thickness of the wall in the S region is 50 to 150 μm, preferably 60-100 μm.

In the R region, the wall 37 of the third tube 47 is made from tubing 8 that is reinforced with a coil or braiding as described above, and is more flexible than the tubing in the S region. Typically the thickness of the wall in the R region is 30 to 100 μm, preferably 50 μm.

In the F region, the wall 37 of the third tube 47 may not be reinforced and may be made from tubing 6 that does not have a coiled or braided wire; the wall of the F region may be thinner than that of the R region. Typically the thickness of the wall in the F region is 30 to 120 μm, preferably 50 μm.

Its location distal of the ATL side port 45 implies that deformation or collapse of the wall will not affect the passage of fluid that travels in the additional lumen proximal to the ATL side port 45. As a consequence, the wall 37 of the third tube 47may be more flexible in the F region than in the R region. The tubing in the F region may be made from any suitable material, including, but are not limited to a polymer such as polypropylene, polyethylene, polyurethanes, polyamide, polyimide, poly(ethylene terephthalate) (PET) or polyesters and copolymers thereof, metal (stainless steel, nitinol) of a combination of metal and polymer.

The regions of tubing adjacent the R region may be joined thereto using an adhesive.

A fluid delivery coupling may be attached to the proximal end 20 of the catheter, which has a guidewire port, and two lateral side ports, one for introduction of inflation medium into the inflation lumen 36 and the other for infusion of fluidic substance into the guidewire lumen 32. The couplings may be provided as a single device, or a two or more separate devices. The each coupling may be a hemostatic valve as described, for instance, in U.S. Pat. No. 5,195,980 and which is incorporated herein by reference. Where the catheter is provided with the aforementioned additional lumen, it will be appreciated that the coupling is provided with an additional lateral side port for the introduction or aspiration of fluidic substance.

The hemostatic valve and the luer lock may be integrated in one single connector. The hemostatic valve allows closing temporarily the guide wire lumen of the OTW catheter at its proximal end and the expansion member allows closing the guide wire lumen at its distal end. This allows, when applying positive pressure to the inflation lumen 36, to exert efficiently pressure on the expansion member and to close the distal end. The hemostatic valve has a lateral luer access allowing for injection of a liquid inside the guide wire lumen which will come out through side ports 41.

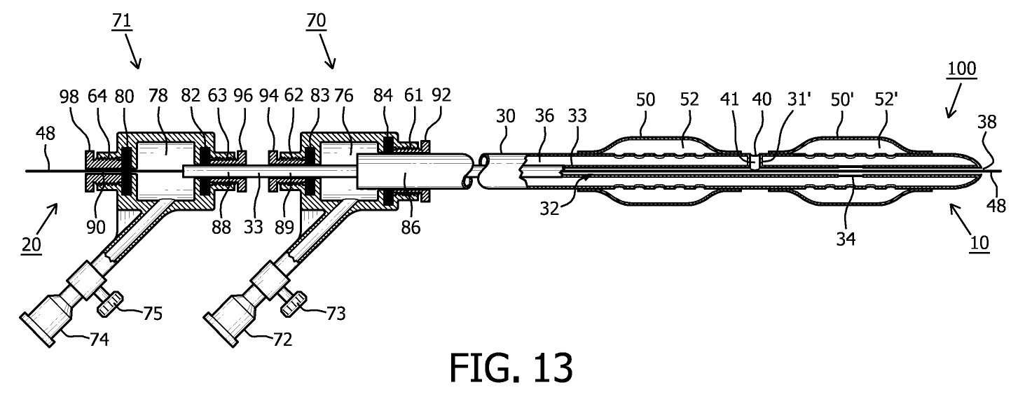

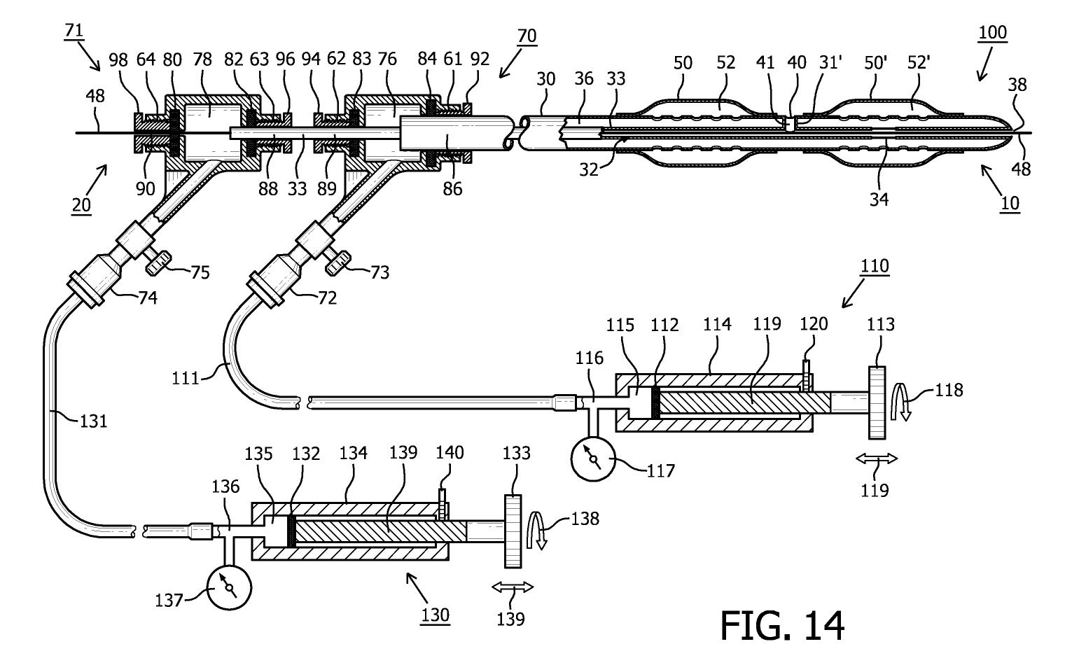

According to one aspect, the invention relates to a pair of fluid delivery couplings 70, 71 (FIGS. 13 to 15, each configured to couple the proximal end of the catheter 20 to a fluid pump 110, 130 (FIG. 14), one to allow inflation of the balloon 50, 50′ and the other for the delivery of fluidic substance. It allows access to the open proximal end 20 inflation lumen 36 and guidewire lumen 32.

The distal fluid delivery coupling 70 allows inflation of the balloon 50, 50′ through the inflation lumen 36 while the proximal fluid delivery coupling 71 allows the delivery of fluidic substance through the guidewire lumen 32.

With reference to FIG. 13 the distal fluid delivery coupling 70 may comprise a distal port 86 disposed with an O-ring seal 84, and a proximal port 89 also disposed with an O-ring seal 83. A chamber 76 is in fluid connection with the distal port 86, the proximal port 89, and a distal pump connector 72. The pump connector 72 is preferably operably connected to a valve 73. The distal port 86 is configured to receive the proximal end of the catheter shaft 30, and can form a seal against the outer wall of the shaft 30. The proximal port 89, preferably of a narrower diameter than the distal port 86, is configured to receive the cylindrical wall 33 of the guidewire lumen and can form a seal against the wall 33 of the guidewire lumen 32 distal to its opening. The proximal port 89, may not accept the wider diameter of the shaft 30; a consequence is that the proximal end of the shaft 30 is located in the chamber 76, in fluid connection with the pump connector 72. When the distal port 86 and proximal port 89 are occupied, a water-tight connection is thus formed between the inflation lumen 36 of the catheter and a pump connector 72 for connection to a fluid (inflation) pump 110 (FIG. 14). The pump connector 72 may be a screw connection, push-fit connection, a Luer connection or other suitable coupling.

With reference to FIG. 13 the proximal fluid delivery coupling 71 also comprises a distal port 88 disposed with a O-ring seal 82, and a proximal port 90 also disposed with an O-ring seal 80. A chamber 78 is in fluid connection with the distal port 88, the proximal port 90, and a proximal pump connector 74. The pump connector 74 is preferably operably connected to a valve 75. The distal port 88 accepts the proximal end of the cylindrical wall 33 of the guidewire lumen 32, and can form a seal against said cylindrical wall 33. The proximal port 90, preferably of a narrower diameter than the distal port 88, accepts the guidewire 48 and can form a seal against the guidewire. The proximal port 88, may not accept the wider diameter of the cylindrical wall 33 of the guidewire lumen 32; a consequence is that the proximal end of said cylindrical wall is located in the chamber 78, in fluid connection with the pump connector 74. When the distal port 88 and proximal port 90 are occupied, a water-tight connection is thus formed between the guidewire lumen 32 of the catheter and a pump connector 74 for connection to a fluidpump 130 (FIG. 14). The pump connector 74 may be a screw connection, push-fit connection, a Luer connection or other suitable coupling.

One embodiment of the invention is a distal fluid delivery coupling 70 comprising:
a distal port 86, configured to accept the proximal end of the shaft 30 and form a seal against the body of said shaft 30,
a proximal port 89 configured to accept the guidewire lumen, and to form a seal against the wall 33 of the guidewire lumen, and
a pump connector 72, configured to connect to an inflation pump 110;
   which ports 86, 89 and pump connector 72 are in fluid connection with a chamber 76 in the coupling 70 which accepts the proximal end of the shaft 30.

One embodiment of the invention is a proximal fluid delivery coupling 71 comprising:
a distal port 88, configured to accept the proximal end of guidewire lumen and form a against the wall 33 of the guidewire lumen,
a proximal port 90 configured to accept the guidewire 48, and to form a seal against the guidewire 48, and
a pump connector 74, configured to connect to an inflation pump 130;
   which ports 88, 89 and pump connector 74 are in fluid connection with a chamber 78 in the coupling 71 which accepts the proximal end of the guidewire lumen.

Another embodiment of the invention is distal fluid delivery coupling 70 comprising:
a distal port 86, disposed with a distal seal 84,
a proximal port 89 disposed with a proximal seal 82, and
pump coupling 72 operably connected to a valve 73;
   which are in fluid connection with a chamber 76 in the coupling 70, wherein the
distal port 86 is configured to accept the proximal end of the shaft 30 and form a seal against the body of said shaft 30, and
proximal port 89 configured to accept the guidewire lumen, and to form a seal against the wall 33 of the guidewire lumen, allowing the proximal end of the shaft 30 to pass through the coupling 70.

Another embodiment of the invention is proximal fluid delivery coupling 71 comprising:
a distal port 88, disposed with a distal seal 82,
a proximal port 90 disposed with a proximal seal 80, and
pump coupling 74 operably connected to a valve 75;
   which are in fluid connection with a chamber 78 in the coupling 71, wherein the
distal port 88 configured to accept the proximal end of guidewire lumen and form a seal against the wall 33 of the guidewire lumen, and
proximal port 90 is configured to accept the guidewire 48, and to form a seal against the guidewire 48, allowing the proximal end of the guidewire lumen to pass through the coupling 71.

As mentioned above, each fluid delivery coupling 70, 71 (FIG. 13) comprises the distal port 86, 88 disposed with a distal seal 84, 82 and the proximal port 89, 90, disposed with a proximal seal 83, 80.

The distal seal 84, 82 and proximal seal 83, 80 are preferably compressible annular rings whose inside diameter can be reduced by the application of a compression force parallel to the central axis of the ring. This might be achieved, for example, by providing a threaded extension 61, 62, 63, 64 to each port 86, 88, 89, 90 to which a threaded bolt 92, 94, 96, 98 can engage (FIG. 13). Said bolt has a hollow shaft and head through which the catheter shaft 30, guidewire lumen wall 33, or guidewire 48 can pass. Tightening the bolt 92, 94, 96, 98 results in compression of the respective seals 84, 83 82, 80 and, a sealing of the ports 86, 89, 88, 90 against shaft 30, guidewire lumen wall 33, or guidewire 48, respectively.

A central axis of the distal port 86, 88 and proximal port 89, 90 are preferably essentially aligned i.e. coaxially aligned. This allows the guidewire 48 to pass though all the ports without kinking. According to one aspect of the invention, the inflation coupling 12, is a double Y-shape coupling.

Thus, the distal fluid delivery coupling 70 fluidly connects the inflation lumen 36 of the catheter 100 and the distal pump connector 72, by forming a distal chamber 76 sealed by the outside surface of the shaft 30 at the proximal end 20 and by the outside surface of guidewire lumen 33 distal to its opening. The proximal fluid delivery coupling 71 fluidly connects the guidewire lumen 36 of the catheter 100 and the proximal pump connector 74, by forming a chamber 78 sealed by the outside surface of the wall 33 of the guidewire lumen at the proximal end 20 and by the outside surface of guidewire 38.

The distal pump connector 72 may be disposed with a valve (tap) 73 to maintain pressure in the inflation lumen 36 after the inflation pump 110 has been disconnected. Thus the balloons 50, 50′ remain inflated when the valve 73 is closed. The proximal pump connector 74 may also be disposed with a valve (tap) 75.

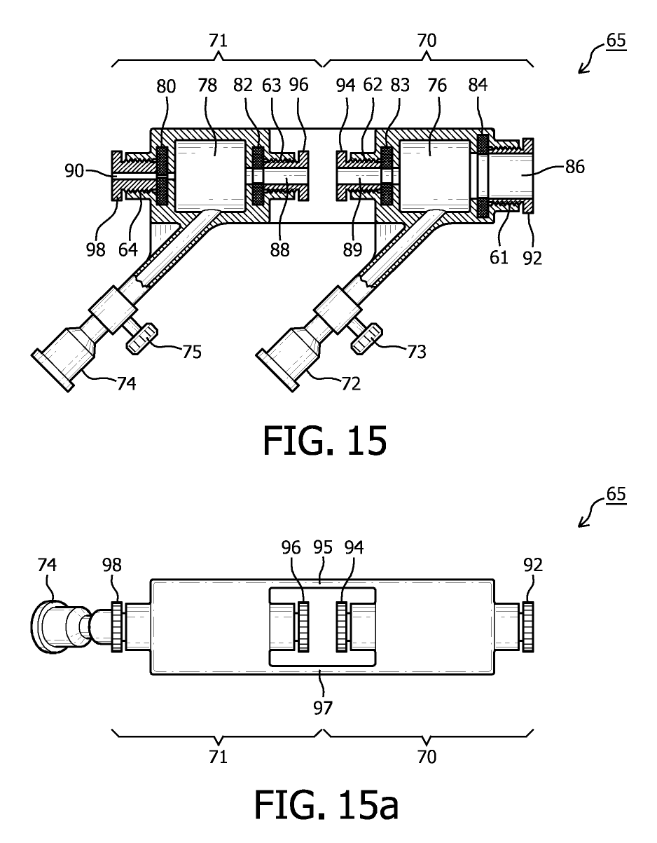

While the distal and proximal fluid delivery couplings 70, 71 are show as separate entities in FIGS. 13 and 14 it is within the scope of the invention that they are joined as a single device 65, as shown for example, in FIG. 15. In FIG. 15, the distal and proximal fluid delivery couplings 70, 71 are tandemly arranged and rigidly joined by two bridging elements 95, 97 (FIG. 15a) to form a single device 65. A central axis of the distal ports 86, 88 and proximal ports 89, 90 are preferably essentially aligned i.e. coaxially aligned. This allows the guidewire 48 to pass though all four ports without kinking.

The distal and proximal fluid delivery couplings 70, 71 or single entity 65 formed therefrom may be provided in a kit along with the catheter 100.

Another aspect of the invention is a fluid pump 110, 130, more specifically, a pair of fluid pumps. Such a pump 110 provides pressure of inflation fluid to the catheter 100 allowing gradual inflation and deflation of the balloon and contraction and closure of the expansion member 34. Alternatively, such pump 130 provides pressure for delivery of fluid substance along the guidewire lumen 32. These pumps 110, 130 are well known in the art.

Generally a fluid pump is a syringe-type arrangement, whereby the distance moved by a plunger element can be finely controlled by the practitioner and whereby the pressure applied by the fluid can be monitored by means of a pressure gauge. An embodiment of a fluid pump 110, 130 according to the invention is depicted in FIG. 14; two pumps are shown, one 110 connected to a distal pump connector 72 via tubing 111, the other 130 connected to a proximal pump connector 72 via tubing 131. Each pump 110, 130 comprises a plunger 112, 132 that is able to move linearly in a housing 114, 134, changing the volume of a water-tight chamber 115, 135 at the distal end. The chamber exits at an outlet port 116, 136, and is fluidicly coupled to a pressure meter 117, 137. An outlet port 116, 136 is connected by a tubing 111, 131 to the pump connector 72, 74 of the fluid delivery coupling 70, 71. The plunger 112, 132 is operated by a handle 113, 133. For a fine control, the handle 113, 133 may be turned 118, 138 and a threaded shaft 119, 139 of the plunger 112, 132 advances or withdraws linearly according to the direction the handle 113, 133 is rotated. For a coarse adjustment, the handle 113, 133 may be pushed or pulled 119, 139 to advance or withdraw the plunger 112, 132 directly. The rotation or push/turning modes of operation may be selected by a button 120, 140 on the side of the housing which controls the engagement of the thread with the housing 114, 134. Such pumps are well known in the art, for example, manufactured by Boston Scientific.

The fluid pumps 110, 130 may be provided in a kit along with the catheter 100 and optionally fluid delivery couplings 70, 71.

The catheter of the invention may be provided with at least one inflatable balloon 50, 50′. at the distal end 10, the inflation lumen 36 being in fluidic connection with the balloon lumen 52. Typically, a plurality of openings 53, 53′ in the shaft 30 bring the inflation lumen 36 into fluid connection with the balloon lumen 52. The openings may be replaced with a gap in the shaft 30 as shown, for instance, in FIGS. 21 and 22.

The inflatable balloon 50, 50′ in the uninflated condition typically comprises a plurality (e.g. 2, 3, 4, 5, 6) of folded wings, folded in any fashion, preferably folded around a central longitudinal balloon axis as is well known in the art, to form the narrow cylindrical balloon profile. As is well understood, the wing structure is formed from the balloon in a flattened condition, each wing extending from the outer radial balloon edge towards the central axis. Prior to folding, the wings may be radially extending, spaced from one another in the circumferential direction around the central longitudinal axis of the balloon. A wing in the folded condition is generally devoid of inflation medium, gaseous or fluid.

The inflatable balloon 50, 50′ is suitable for insertion into a cavity, which after insertion and inflation at least partly contacts the cavity wall of a subject for the delivery of composition. Various types of balloon are known with a plurality of shapes and features suited, after inflation, to the cavity shape and treatment regime. For example, a balloon after inflation may be longitudinal, ovoid, conical, cylindrical, barrel, hour-glass, bullet shaped or any shape that can accommodate the cavity receiving treatment.

In preparing the inflatable balloon 50, 50′ according to the invention, the uninflated balloon is arranged, depending on the size of the balloon, into 2, 3, 4, 5, or 6 wings, in a manner known per se to provide a propeller-type profile. The wings are folded in a clock-wise or anti-clockwise direction. The folded balloon so formed has compact and narrow profile that makes it possible to guide the balloon catheter through vessel and lumina. While the above provides a general guidance, the skilled person will understand the routine variations and adaptations that can be readily implemented; these variations also fall under the scope of the invention.

The wings of the inflatable balloon 50, 50′ may be maintained in the folded condition by dint of a substance having light adhesive property present in the composition, or disposed over the wing edges. Alternatively, the folded wings may be subjected to a heat and/or pressure treatment to maintain their structure, the parameters of which will depend on the lability of the composition. Alternatively, the folded state may be maintained by introducing a relief structure as described for instance in US 2003/0014100 and US 2003/0014070, and elaborated further below.

The balloon may be uncoated, coated, for example, with medicament, or provided with a radially expanding implant such as a stent.

The inflatable balloon 50, 50′ may be configured to expand essentially simultaneous with the expansion member 34. Inflatable balloon 50, 50′ pressure can be modulated according to the thickness and material of the balloon. The inflatable balloon 50 is formed from any suitable expandable material. Examples of suitable materials include latex rubber, polyamide 11 or 12, PET, polyurethane, or any material known by any skilled in the art.

Where more than one balloon 50, 50′ is provided, the balloons are tandemly arranged in longitudinal displacement along the shaft 30. The inflation lumen 36 is in fluidic connection with the lumens 52, 52′ of each balloon 50, 50′. As mentioned elsewhere, the one or more adjacent pairs of balloons may flank a TL-side port 41 which arrangement allows fluid medicament to be delivered to a treatment region sealed between two balloons, preventing systemic circulation of the medicament.

According to one aspect of the invention, when more than one balloon 50, 50′, 51 is provided, the distal most balloon 51 (FIG. 6) is shorter and is configured to inflate to shorter length compared with the other balloons 50. Preferably, the TL side port 41 is provided adjacent and proximal to the distal most balloon 51. Such arrangement allows the passage of fluidic substance in both proximal and distal directions. In this particular case, the area of the vessel that would be treated would be preferably the area corresponding to the balloon located in the middle, between a long and short balloon (FIG. 6).

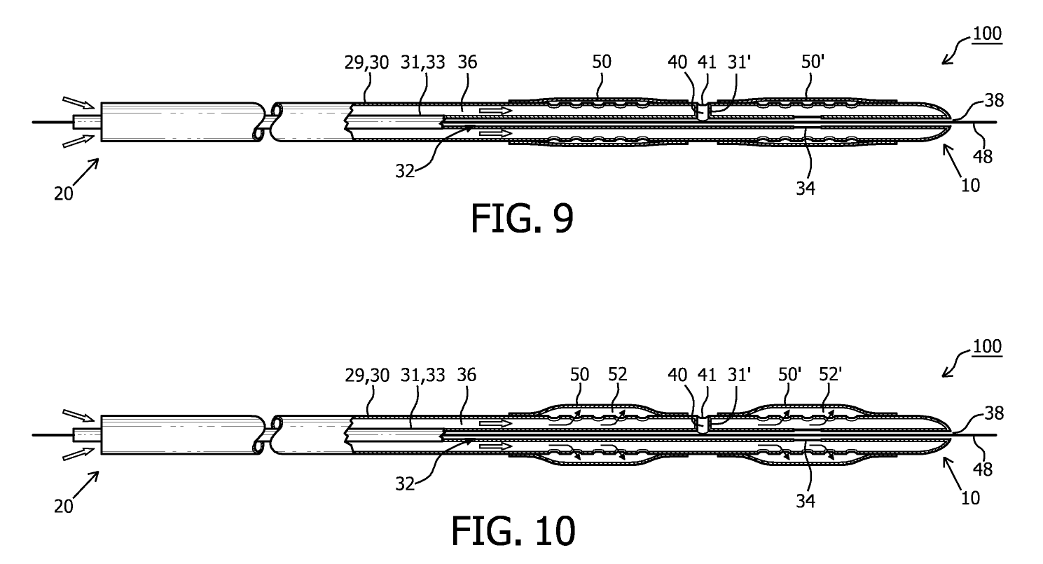

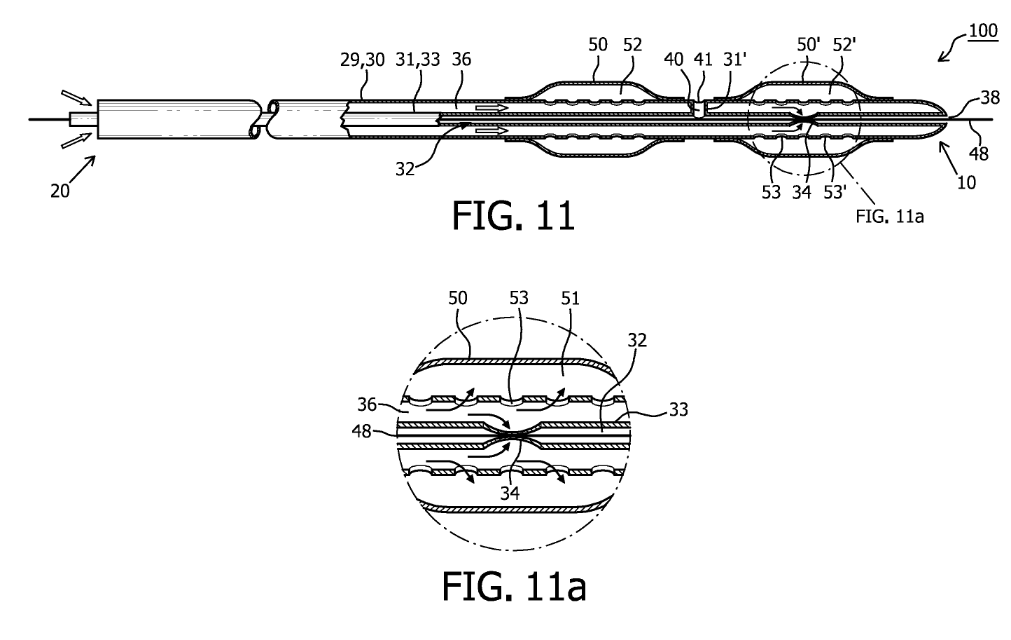

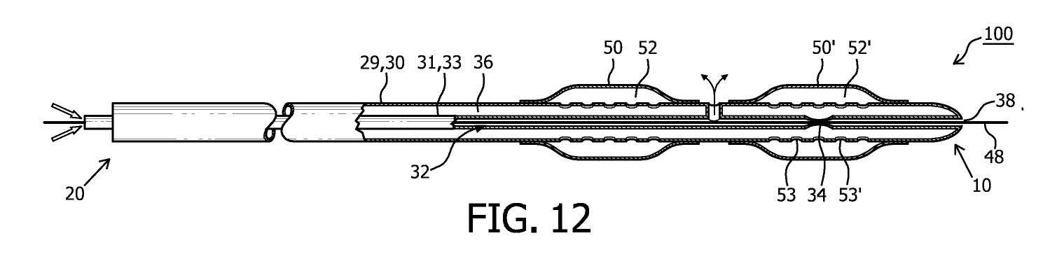

As mentioned, the present invention provides for the delivery of a fluidic substance to the site of treatment from the guidewire lumen 32 to a side port 41 in the distal end 10 of the catheter 100. One possible procedure is illustrated in a series of figures (FIGS. 9 to 12), in which the balloon catheter 100 of the invention is advanced into a vessel (not shown) of the subject until it has been properly positioned i.e. the distal end is adjacent to the site of treatment. Inflation medium (e.g. saline or 50% saline mixed with 50% contrast medium) is introduced into the inflation lumen 36 (FIG. 9), via the proximal end. In an initial step, the balloons 50 inflate (FIG. 10) until they reach a maximum expansion limit. At the same time, the expansion member 34 expands to the extent that it occludes the guidewire lumen 32 at the distal 10 end (FIG. 11, FIG. 11a). The guidewire lumen 32, sealed from the distal port 38, receives a fluidic substance introduced through the proximal end, which substance exits the guidewire lumen 32 through the GL-side port 41 and into the target area (FIG. 12). Inflated balloons 50 and 50′ which flank each GL-side port 41 seal the target area, focusing the exposure site, and preventing systemic contamination.

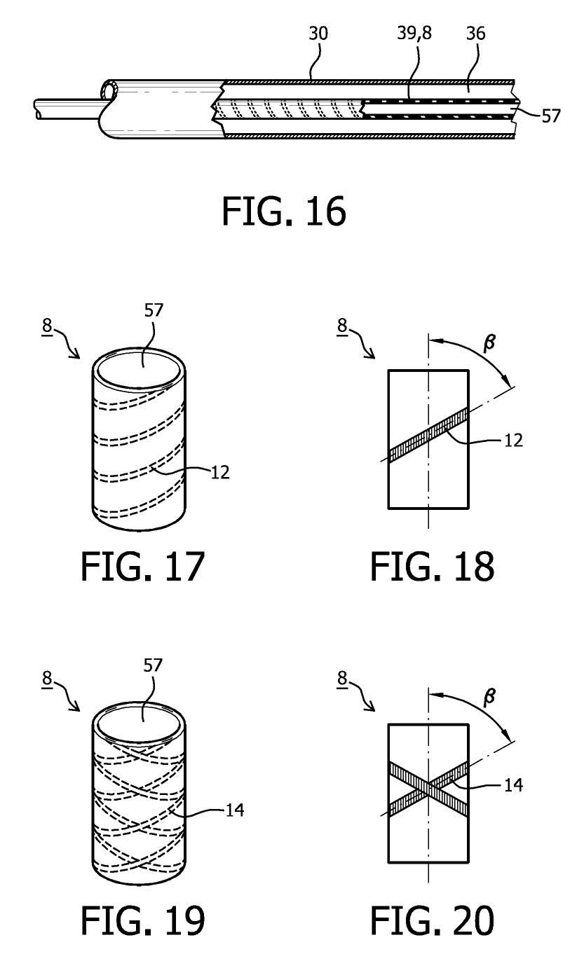

One embodiment of the invention, with reference to FIG. 16 concerns a catheter 100 having a proximal end 20 and distal end 10, comprising an elongated longitudinal shaft 30, which forms the wall of an inflation lumen 36 for one or more balloons, and an inner lumen 57 for the passage of a guidewire and/or fluidic substance disposed within the inflation lumen and fluidicly isolated there from. It will be obvious that the inflation lumen is contained within a second tube while the inner lumen is contained within a first tube, the first (inner) tube residing within the inflation lumen of the second tube. The outer surface of the wall 39 of the first tube 57 is in fluidic contact with the inflation lumen 36

At least part, optionally all, of the wall 39 of the first tube is made from reinforced tubing 8. The reinforced tubing 8 reduces deformation of the wall 39 of the first tube 57 when hydrostatic pressure is applied to the inflation lumen 36 thereby maximizing its transverse cross-sectional area for the passage of fluid or guidewire. The tubing is reinforced using a coiled wire 12 (FIG. 17) or braided wire 14 (FIG. 19) disposed in the longitudinal direction of the tubing. The coil has a helical path; it may be formed from a single helix or more than one helix. The braiding typically has a cross-cross pattern, formed from two helices of wire running in opposite directions. The inventors have surprisingly found that the reinforcing wire is most effective when it adopts a helix angle of 60 deg or more. Preferably, at least one strand of the coil or braiding has a helix angle of 60 deg or more.

The wall of the tubing 8 is made from any suitable material polymeric material such as polyamide or polyimide, preferably polyimide. The reinforcing coiled wire 12 or braided wire 14 may be made from any material having suitable tensile strength such as stainless steel, nitinol, phynox, silver. The wire of the coil or braiding is provided within the tubing wall, or on the outside or inside of the wall. The reinforced tubing may be prepared by depositing the polymeric material over the coiled or braided wire; deposition allows a more accurate control over the thickness of the reinforced tube wall. Examples of commercially available reinforced tubing include for instance polyimide coiled tubes produced by Microlumen.

The helix angle is the angle between a helix of the coiled of braided wire the central axial of the tubing 8 as shown, for example, in FIGS. 18 and 20. The term is well understood in the art. The helix angle may be 60, 65, 60, 75, 80, 85, 90, 95 deg or more, or a value in the range between any two of the aforementioned values, preferably more than 60 deg, more preferably between 60 and 90 deg.

The reinforced tubing 8 may form part or optionally all of the inner lumen of any catheter. The catheter may exist in the art or may be a future catheter.

The reinforced tubing 8 may form part, optionally all of a guidewire lumen having an expandable member as described throughout the text herein, and as illustrated in FIGS. 21, 22, and 24.

The reinforced tubing may form part, optionally all of the additional third tube 47 (FIG. 24) of a catheter described herein.

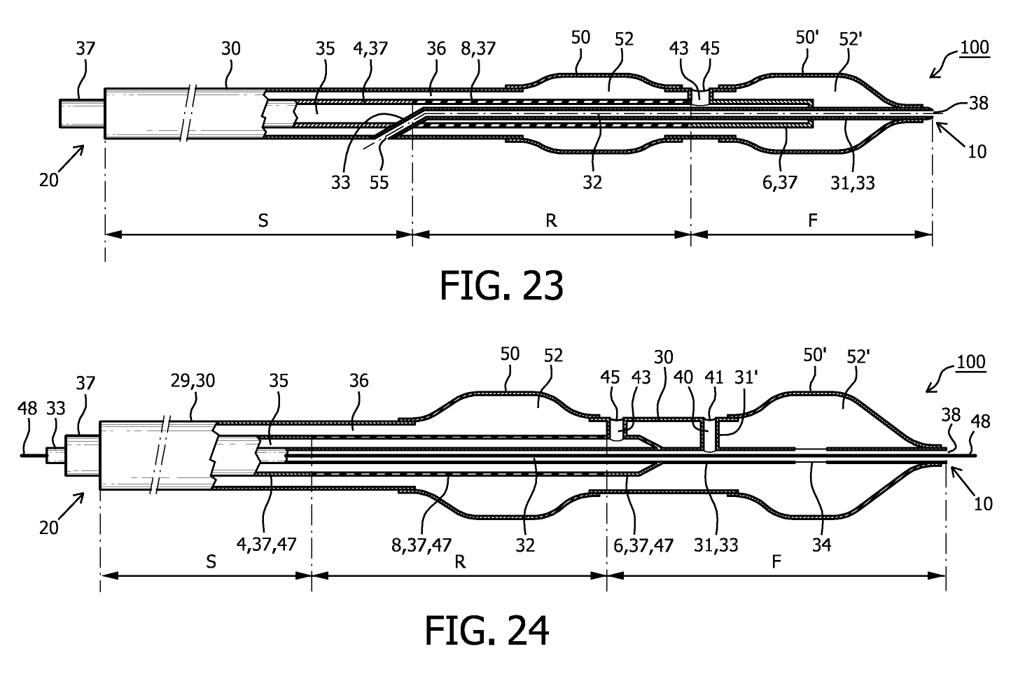

The reinforced tubing may form part, optionally all of the additional third tube 47 (FIG. 24) of a catheter described herein, adapted so that the guidewire lumen 32 operates in a rapid exchange mode; an instance of this embodiment is depicted in FIG. 23. According to this embodiment,
the first tube 31 is devoid of an expansion member 34,
the proximal end of the guidewire lumen 32 exits through a guidewire side port 55 in the side wall of the shaft 30 situated towards the distal end of the catheter,
the guidewire lumen 32 is devoid of the TL lumen 41 and side port 40,
at its distal end 10, the additional lumen 35 is closed and fluidicly sealed against the outer wall 33 of the guidewire lumen 32 towards the distal end, and distal to the ATL side port 45.

1. A catheter comprising:
a proximal end, a distal end, and an elongated shaft containing:
a longitudinal first tube provided with a guidewire lumen terminating in a distal terminal port in the shaft, said guidewire lumen being configured for an over-the-wire or rapid-exchange mode of operation, a wall of the first tube being provided with an expansion member of expandable material that is a sub-region of the first tube wall
an inflation lumen extending from the proximal end towards the distal end of the shaft for fluidic contact with the expansion member, said expansion member being configured to expand or contract responsive to pressure in the inflation lumen; and
a transverse lumen, TL, defined by an transverse tube, TT, proximal to the expansion member, fluidicly connecting the guidewire lumen to a TL-side port on a side wall of the shaft.

2. Catheter according to claim 1, wherein the shaft comprises:
at least one inflatable balloon at the distal end, the inflation lumen being positioned for fluidic connection with a balloon lumen.

3. Catheter according to claim 2, wherein:
the TL-side port is located proximal to a most proximal inflatable balloon, or
wherein a number of the inflatable balloons is two or more, and any two balloons flank a TL side port.

4. Catheter according to claim 1, wherein the expansion member is located distal to the TL-side port.

5. Catheter according to claim 1, wherein the guidewire lumen, when configured for an over-the-wire mode of operation, will extend to the proximal terminal end of the shaft.

6. Catheter according to claim 1, wherein the guidewire lumen, when configured for a rapid exchange mode of operation, is branched,
a side branch being provided for the passage of a guidewire through a GL (guidewire) side port in side wall of the shaft, and
a longitudinal branch extending to a termination of the proximal end of the shaft, configured for the passage of fluid, to an exclusion of the guidewire.

7. Catheter according to claim 6, comprising:
an additional expansion member, said additional expansion member being located on the side branch.

8. Catheter according to claim 1, wherein the inflation lumen is defined by a second tube that is the shaft.

9. Catheter according to claim 1, wherein at least part of the wall of the first tube is made from tubing reinforced with a helically coiled wire or helically braided wire having a helix angle of 60 degrees or more, which tubing is resistive to radial pressure applied in the inflation lumen.

10. Catheter according to claim 9, wherein the wall of the first tube comprises:
two regions of tubing of differential stiffness in the a longitudinal direction:
an R-region containing said reinforced tubing; and
an F-region distal to the R-region, and containing tubing that is more flexible than that in the R-region, and optionally devoid of the coiled wire or braided wire.

11. Catheter according to claim 10, wherein the wall of the first tube comprises:
a further region of tubing of differential stiffness, that is an S-region proximal to the R-region, containing tubing that is less flexible than that in the R-region, and is optionally devoid of the coiled wire or braided wire.

12. Catheter according to claim 1, comprising:
an additional inner lumen defined by a tube third tube, wherein the additional inner lumen is
fluidicly isolated from the inflation lumen and the guidewire lumen;
concentrically arranged around part of the guidewire lumen proximal to the TL side port and to the expansion member;
closed and fluidicly sealed, at its distal end, against the outer wall of the guidewire lumen; and
connected via an additional transverse lumen ATL, to at least one ATL-side port on the side wall of the shaft.

13. Catheter according to claim 12, wherein the wall of the third tube comprises:
two regions of tubing of differential stiffness in the longitudinal direction,
an R-region containing said reinforced tubing; and
an F-region distal to the R-region, and containing tubing that is more flexible than that in the R-region, and optionally devoid of the coiled wire or braided wire.

14. Catheter according to claim 13, wherein the wall of the third tube comprises:
a further region of tubing of differential stiffness, that is an S-region proximal to the R-region, containing tubing that is less flexible than that in the R-region, and is optionally devoid of the coiled wire or braided wire.

15. Catheter according to claim 3, wherein the expansion member is located distal to the TL-side port.

16. Catheter according to claim 15, wherein the guidewire lumen, when configured for an over-the-wire mode of operation, will extend to the proximal terminal end of the shaft.

17. Catheter according to claim 16, wherein the guidewire lumen, when configured for a rapid exchange mode of operation, is branched,
a side branch being provided for passage of a guidewire through a GL (guidewire) side port in side wall of the shaft; and
a longitudinal branch extending to a termination of the proximal end of the shaft, configured for passage of fluid, to an exclusion of the guidewire.

18. Catheter according to claim 17, wherein the inflation lumen is defined by a second tube that is the shaft.

19. Catheter according to claim 18, wherein at least part of the wall of the first tube is made from tubing reinforced with a helically coiled wire or helically braided wire having a helix angle of 60 degrees or more, which tubing is resistive to radial pressure applied in the inflation lumen.

20. Catheter according to claim 19, comprising:
an additional inner lumen defined by a tube third tube, wherein the additional inner lumen is
fluidicly isolated from the inflation lumen and the guidewire lumen;
concentrically arranged around part of the guidewire lumen proximal to the TL side port and to the expansion member;
closed and fluidicly sealed, at its distal end, against the outer wall of the guidewire lumen; and
connected via an additional transverse lumen ATL, to at least one ATL-side port on the side wall of the shaft.
